(19) United States
(12) Patent Application Publication
(10) Pub. No.: US 2009/0192604 A1
(43) Pub. Date: Jul. 30, 2009

(54) Sizer, Holder and Delivery Devices for Minimally Invasive Cardiac Surgery

(75) Inventor: Michael A. Gloss, Minneapolis, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/358,872

(22) Filed: Jan. 23, 2009

Related U.S. Application Data
(60) Provisional application No. 61/062,414, filed on Jan. 25, 2008.

(51) Int. Cl. A61F 2/24

(52) U.S. Cl. 623/2.11; 623/2.36

(57) ABSTRACT

Described is a device for delivering a valve annulus sizer or annuloplasty device holder to a heart valve annulus, the device comprising: an elongate shaft comprising a proximal end, a distal end and at least a portion of the shaft being malleable, wherein the distal end of the elongate shaft connects to the sizer or holder. Also described are a sizer device for sizing a heart valve annulus, and a delivery device that releasably attaches a sizer device or a holder device for delivery to and/or from a heart valve annulus.

### PRIORITY

The present non-provisional patent application claims benefit from United States Provisional Patent Application having Ser. No. 61/062,414, filed on Jan. 25, 2008, by Kuehn et al., and titled SIZER, HOLDER AND DELIVERY DEVICES FOR MINIMALLY INVASIVE ANNULOPLASTY SURGERY, wherein the entirety of said provisional patent application is incorporated herein by reference.

### TECHNICAL FIELD

This invention generally relates to devices and methods of repair and replacement of heart valves. In particular, the invention relates to devices for measuring the size of a heart valve annulus and for holding and delivering an annuloplasty device to the annulus during minimally invasive cardiac surgery.

### BACKGROUND OF THE INVENTION

Heart valve disease is a widespread condition in which one or more of the valves of the heart fails to function properly. Various surgical techniques may be used to replace or repair a diseased or damaged valve. Damaged leaflets of the valve may be excised and the annulus sculpted to receive a replacement valve. Another less drastic method for treating defective valves is repair or reconstruction by annuloplasty, in which the effective size of the valve annulus is contracted and reinforced, by attaching a prosthetic annuloplasty ring or band to an interior wall of the heart around the valve annulus. The annuloplasty ring or band is designed to support the functional changes that occur during the cardiac cycle, while maintaining leaflet coaptation and valve integrity.

To perform successful valve replacement and annuloplasty surgeries, the size of the valve annulus must be accurately measured. Sizing may be achieved by measuring the width and the height of the anterior leaflet of the mitral valve, for example, using sizing obturators. Another way to size the annulus is to use valve sizers, which resemble the shape of the valve annulus and are provided in various sizes. In order to use valve sizers, a surgeon estimates the valve annulus size and selects a sizer accordingly. The sizer is guided into proximity of the annulus with a handle. If the sizer is not judged to be the correct size, it is withdrawn, and replaced by a different sizer. Once the size of the annulus has been determined, a properly sized valve or annuloplasty device may be selected. The selected annuloplasty device is on a holder that is delivered to the annulus in order to implant the device.

Surgical techniques for annuloplasty surgery are typically performed open-chest. This usually requires the patient to be placed on a cardiac bypass machine to pump and oxygenate the blood while the surgeon operates on the stopped heart muscle. Open-chest surgery can be very traumatic on the patient and recovery can take many months. Additionally, such surgery may not be an option for some patients due to limited possibility for recovery, concurrent disease, or age.

For these reasons, it is desirable to use minimally invasive cardiac surgical techniques for valve repair. However, these procedures reduce the available space to deliver surgical instruments to a surgical site, and reduce the space in which surgical instruments may be operated within the area of the surgical site. Therefore, such procedures require surgical instruments with appropriate size and maneuverability that accommodate the limited space.

Traditional annuloplasty and valve sizing and holding instruments were designed for use with open-chest surgery that exposes the appropriate regions of the heart to complete and open access through the open chest wall. The ability of these instruments to fit through significantly reduced surgical field access points was not a necessary criteria for their design. Advances in the surgical field toward minimally invasive techniques has created significant new challenges for the design of new instruments and the development of new techniques for using these instruments to successfully complete procedures in limited access surgical fields.

Exemplary types of minimally invasive cardiac surgery include atrio-ventricular valve repair, reconstruction, or replacement surgical procedures. In particular, the replacement of the valves and repair of valve annulus dilation using annuloplasty devices can employ minimally invasive techniques.

Despite the current existence of sizing devices for sizing a valve annulus and holding devices for holding annuloplasty devices, there is still a need for improved devices, and in particular those devices that may be used during minimally invasive cardiac surgical procedures.

### SUMMARY OF THE INVENTION

Embodiments of the present invention include sizer devices, holder devices, and delivery devices that are made, configured and/or may be manipulated to fit through significantly reduced surgical field access points and may be used in reduced surgical fields of operation. The embodiments of the present invention offer an advantage that they may be used during minimally invasive cardiac surgery to fit through significantly reduced surgical field access points and in reduced surgical fields of operation. In doing so, the embodiments of the present invention reduce the physical trauma to the patient by eliminating the need to perform a complete stemotomy, and reduce the time spent in surgery. The embodiments of the present invention also allow annuloplasty surgery to be performed on patients that would not otherwise be able to have the surgery, particularly involving open-chest techniques.

A first aspect of the present invention is a device for delivering a valve annulus sizer or annuloplasty device holder to a heart valve annulus. One embodiment of the device comprises: an elongate shaft comprising a proximal end, a distal end and at least a portion of the shaft being malleable, wherein the distal end of the elongate shaft connects to the sizer or holder. The shaft may be malleable allowing the shaft to obtain a bent configuration that allows the sizer or holder to be delivered to the valve annulus through a minimal access port in a patient's body. The shaft may have a varying cross-section with the malleable portion having a decreased cross-section. The malleable portion of the shaft may comprise a malleable material. The malleable material may comprise Nitinol™. The malleable portion of the shaft may be at or near the distal end.

A second aspect of the present invention is a sizer device for sizing a heart valve annulus. An embodiment of the sizer device may comprise: a sizing plate; and a stem connected to the sizing plate provided for connection of the sizer device to a delivery device, wherein the stem is bendable. The stem may comprise a malleable material. The stem may comprise a joint.

A third aspect of the present invention is a holder device for holding an annuloplasty device for delivery to a heart valve annulus. An embodiment of the holder device may comprise: a holding plate; and a stem connected to the holding plate provided for connection of the holder device to a delivery device, wherein the stem is bendable. The stem may comprise a malleable material. The stem may comprise a joint.

A fourth aspect of the present invention is a delivery device that releasably attaches a sizer device or a holder device for delivery to and/or from a heart valve annulus. One embodiment of the device may comprise: an elongate shaft having a proximal end and a distal end; and a socket extendible from and retractable into the distal end of the shaft, wherein the socket is extended from the distal end of the elongate shaft in order to release or surround the sizer device or the holder device and the socket is retracted into the distal end of the elongate shaft in order to attach the sizer device or the holder device to the delivery device. The socket may be partially retracted into the distal end of the elongate shaft in order to attach the sizer device or the holder device. The sizer device and the holder device may comprise a flexible or malleable stem connected to a sizing or holding plate, and while partially retracted into the distal end of the elongate shaft, the stem of the sizer device or the holder device may be able to flex or bend.

A second embodiment of the device may comprise: an elongate shaft having a proximal end and a distal end, and a wire running there through; a handle attached to the proximal end of the shaft that is attached to the wire, wherein rotation of the handle moves the wire proximally and distally; and a socket moveable within the shaft and extendible from the distal end of the shaft and connected to the wire, wherein when the wire moves distally the socket moves distally and extends from the delivery device and when the wire moves proximally the socket also moves proximally and grasps a holder device or sizer device in order to attach the holder or sizer device to the delivery device. The device may further comprise a housing that houses the socket when the wire is moved proximally. When the socket is moved proximally, the housing may surround the socket and cause the socket to grasp the holder device or sizer device. The device may further comprise a spring connected to the socket and surrounding the wire that bias the socket to a proximal position. The handle may comprise an interior including threads, and an inner element that is attached to the wire such that rotation of the handle causes the inner element to move distally or proximally within the threads which causes the wire to move distally and proximally, respectively.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of a holder device and a delivery device in combination, in accordance with the present invention, showing the delivery device having a straight configuration;

FIG. 2 is a perspective view of the holder device and delivery device combination of FIG. 1, showing the delivery device having a bent configuration;

FIG. 3 is a perspective view of a sizer device, in accordance with the present invention;

FIG. 4 is a perspective view of a sizer device, in accordance with the present invention;

FIG. 5 is a perspective view of a sizer device and a delivery device in combination, in accordance with the present invention;

FIG. 6 is an exploded view of the distal end of the sizer device and delivery device combination of FIG. 5;

FIG. 7A is a cross-sectional view of the proximal end of the delivery device of FIG. 5 in one configuration;

FIG. 7B is the proximal end shown in FIG. 7A and in another configuration;

FIG. 8 is a perspective view of the distal end of the delivery device of FIG. 5 detached from the sizer device of FIG. 5;

FIG. 9 is a perspective view of the delivery device of FIG. 8 engaging the sizer device of FIG. 8;

FIG. 10 is a perspective view of the delivery device and sizer device of FIG. 9 showing the retraction of the sizing device into the delivery device; and

FIG. 11 is a perspective view of the delivery device and sizer device of FIG. 10 showing the sizing device retracted into the delivery device.

### DETAILED DESCRIPTION OF PREFERRED EMBODIMENTS

Embodiments of the present invention include sizer devices, holder devices, and delivery devices that are made, configured and/or may be manipulated to fit through significantly reduced surgical field access points and may be used in reduced surgical fields of operation. The sizer devices and holder devices are preferably made to be attached and detached from the delivery devices of the present invention. Particularly, the sizer devices, holder devices and delivery devices will be discussed with regard to their use during minimally invasive annuloplasty surgery. During annuloplasty surgery, the purpose of the delivery device is to first deliver a sizer device to a valve annulus that is in need of repair in order to size the annulus, and then after removal of the sizer device from the body and the delivery device, the delivery device is next used to deliver a holder device with an attached annuloplasty device to the valve annulus for implantation of the annuloplasty device. Although the present application specifically addresses minimally invasive annuloplasty surgery, it is contemplated that the present invention or features thereof may be used during other minimally invasive surgical procedures as well.

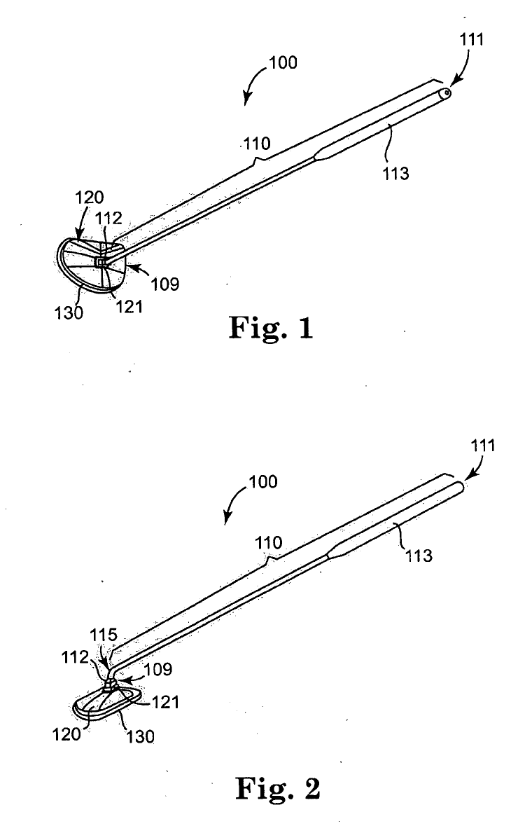

With reference to the accompanying figures, wherein like components are labeled with like numerals throughout the several figures, and, initially, to FIG. 1, one embodiment of the present invention is shown. FIG. 1 shows a device 100 including an elongate delivery device 110 and a holder device 120 in combination. The elongate delivery device 110 has a length such that the holder device 120 may be delivered through a transthoracic window, for example, and to a valve annulus for implantation while a proximal end 111 of the delivery device 110 still remains outside the body. The length of the delivery device 110 is preferably longer than a length of a delivery device used for open-chest annuloplasty surgery.

The elongate delivery device 110 has a proximal end 111 and a distal end 109. Preferably at or near the proximal end 111, the delivery device includes a handle 113 for the purpose of ease in handling the delivery device 110. At the distal end 109 of the delivery device 110 includes a connector 112 that connects or attaches the delivery device 110 to the holder device 120. The connector 112 can either attach permanently or releasably to the holder device 120, and preferably connects to a socket 121 on the holder device 120. The socket 121 preferably accommodates, or may attach to, other holder devices and possibly sizer devices for sizing a valve annulus.

The holder device 120 holds an annuloplasty device 130, for example. The annuloplasty device 130 is attached to the holder device 120 using sutures or other means for attachment. The holder device 120 is preferably shaped and sized to conform to the size and shape of the annuloplasty device 130 being held. One such shape and size is shown in FIG. 1, but others are also contemplated by the present invention.

In order for the holder device 120 and annuloplasty device 130 to be delivered minimally invasively to a desired surgical site in the heart, the delivery device 110 is preferably malleable at or near its distal end 109. The malleability of the delivery device 110 or portion of the delivery device 110, allows the delivery device 110 to obtain the bent configuration shown in FIG. 2. A bend 115 may be possible if the delivery device 110 comprises a malleable material at least in the distal portion and preferably near where the holder 120 is attached to the delivery device 110. Any suitable type of malleable material that would provide such a function is contemplated. One example is Nitinol™ wire.

Alternatively, the cross-section of the elongate delivery device 110 may be decreased towards the distal end 109, which would allow the distal portion to be more malleable than more proximal portions, and would allow the delivery device 110 to include the bend 115 near its distal end 109. A benefit of the delivery device 110 having a variable cross-section is that it would allow the proximal portion to maintain a larger cross-section and a higher level of rigidity near where a user would hold onto the delivery device 110.

As shown in FIG. 2, the delivery device 110 includes the bend 115 near its distal end 109 that preferably allows a major axis of the holder device 120 to extend generally parallel to the elongate delivery device 110. The bend 115 preferably allows the holder device 120, with attached annuloplasty device 130, to fit through a transthoracic window, for example, during minimally invasive surgery on the heart. Preferably, holder device 120 and annuloplasty device 130 are inserted between two ribs, trans-thoracically, and toward the heart using delivery device 110. Preferably, the delivery device 110 is also able to be returned to a straight configuration as needed during such a procedure.

The elongate delivery device 110 would preferably have a length that is sufficient for the holder device 120 to reach its desired location, and preferably by being inserted into the chest cavity trans-thoracically. Although the present invention is disclosed for the purpose of being used during minimally invasive cardiac surgery, other uses are contemplated. For example, the device 100 may alternatively be used in open-chest annuloplasty surgeries. The ability of the delivery device 110 to be bent and straightened may be useful in such open-chest procedures as well.

The delivery device 110 shown in FIGS. 1 and 2 may be used to deliver many different holder devices (120 is one example) and many different sizer devices (not shown) used to size a valve annulus (e.g., a heart valve annulus or a venous valve annulus). An advantage of the delivery device 110 is that it may attach or connect to holder devices and sizer devices that are currently known and already on the market. However, the length of the delivery device 110 and the malleability near the distal end 109 of the delivery device 100 allows an attached holder and/or sizer device to be delivered trans-thoracically.

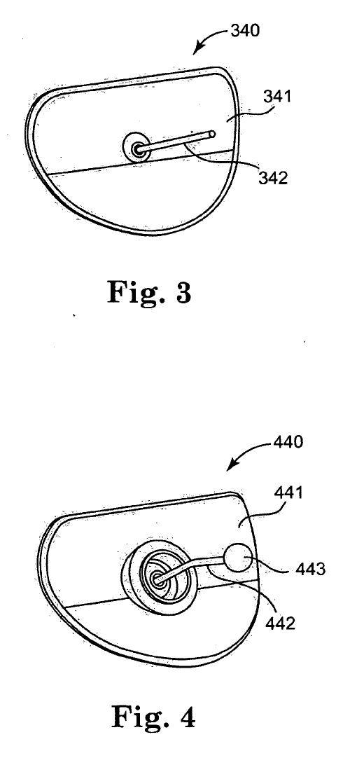

FIG. 3 shows an embodiment of a sizer device 340, in accordance with the present invention. The sizer device 340 could be used with current delivery devices in order to deliver the sizer device 340 to a desired surgical site, for example, trans-thoracically.

The sizer device 340 comprises a sizing plate 341 and a stem portion 342. The sizing plate 341 would preferably be similar to that of current sizing devices. Preferably, the sizing plate 341 would comprise a rigid material that is optically transparent. The sizing plate 341 could be planar, or curved, as shown in FIG. 3. Other two-dimensional and three-dimensional shapes are also contemplated.

The stem 342 of sizing device 340 attaches the sizing plate 341 to a delivery device or handle (not shown). Preferably, the stem 342 would be malleable. A preferred type of material for such a flexible stem portion 342 is a material that is flexible, malleable and also has shape memory. In particular, Nitinol™ is a preferred material, although other materials are also contemplated. The malleable stem 342 could bend in order to allow the sizing plate 341 to be turned so that a major diameter of the sizing plate 341 extends generally parallel to the length of the delivery device (not shown). This would allow the sizing plate 341 to be inserted trans-thoracically, for example, during minimally invasive annuloplasty surgery. The stem 342 would also preferably be able to be straightened after being bent.

The design of sizer device 340 could also be used for a holder device instead. Such a holder device (not shown) could include a malleable stem, like that shown in FIG. 3 and described above. An advantage of using a holder device with such a malleable stem portion is that a delivery device attached to the holder device could deliver the holder device to a surgical site through a transthoracic window.

FIG. 4 shows an embodiment of a sizer device 440, in accordance with the present invention. The sizer device 440 could be used with any suitable delivery device (including, but not limited to, delivery device 510, described below). The sizer device 440 includes a sizing plate 441, a malleable or shape memory or jointed stem 442 and a spherical end 443 on the stem 442. The sizing plate 441 may be similar to that in sizer device 340, and the discussion of sizing plate 341 above also applies to sizing plate 441.

The stem 442 of sizing device 440 is preferably malleable or jointed to allow the sizing plate 441 to be moved or angled while attached to a delivery device (not shown), for example, in order to fit through a transthoracic window or surgical port during minimally invasive surgery. The spherical end 443 is provided for means to attach the sizer device 440 to a delivery device (not shown). As with the sizer device 340 in FIG. 3, the sizer device 440 may alternatively be a holder device having such features.

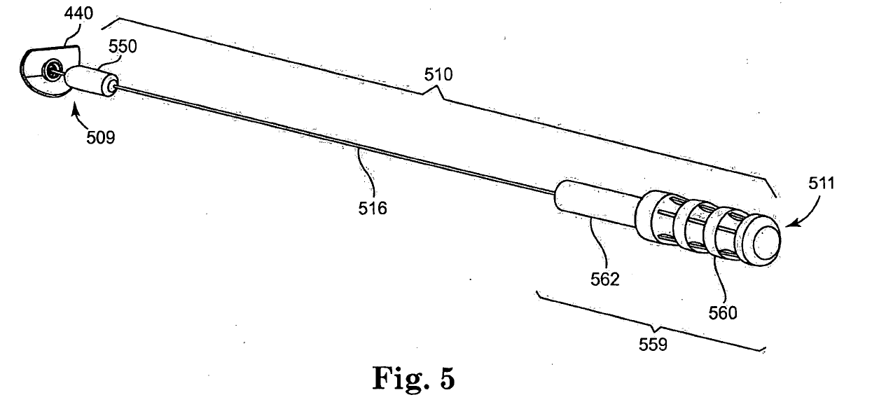

The sizer device 440 of FIG. 4 is preferably used with an elongate delivery device such as that shown in FIG. 5. The elongate delivery device 510 includes a proximal end 511 and a distal end 509. At the distal end 509, the delivery device 510 includes a grasping means 550 (which will be discussed in more detail below). The grasping means 550 preferably has the ability to engage or connect to the stem and/or the spherical end (both not visible in FIG. 5) of sizing device 440, although it is also contemplated that the grasping means 550 may engage or connect to other such similar features of other devices.

The elongate delivery device 510 also includes a handle 559 at its proximal end 511. The handle 559 includes components, which will be discussed below, that control the grasping means 550 remotely. Such controls may be used outside the body of a patient to remotely control the grasping means 550, in order to release and grasp sizing devices or holding devices within the body.

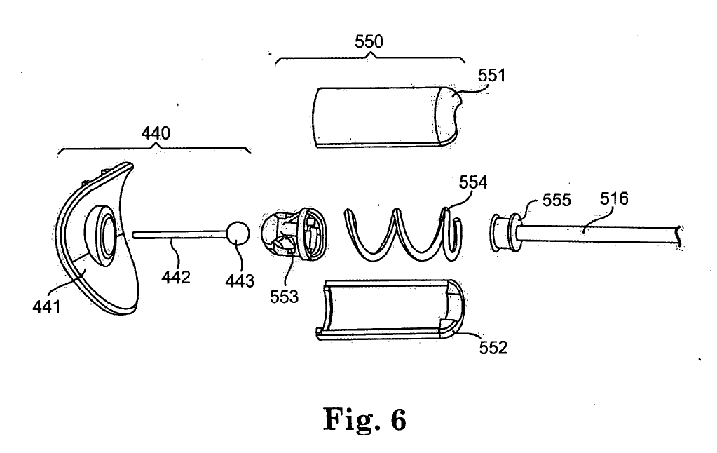

The grasping means 550 is shown in an exploded view in FIG. 6, along with sizing device 440. Grasping means 550 is shown to comprise first 551 and second 552 housing segments for housing a socket 553 and other components. The housing segments 551, 552 may be attached together in order to house the other components of the grasping means 550. The other components preferably include the socket 553, a spring 554, and a connector 555 that is connected to an elongate segment 516 of the delivery device 510. A wire (not shown) runs through elongate segment 516 and is preferably connected to the socket 553 in order to move the socket 553 proximally and distally as desired. The wire (not shown) is controlled remotely from the proximal end 511 of elongate delivery device 510. The spring 554 biases the socket 553 to a distal position within the housing (segments 551, 552).

The socket 553 is comprised of multiple portions that allow the socket 553 to open to engage the spherical end 443 on sizing device 440 while extended from the housing formed by the housing segments 551, 552. When the socket 553 is retracted into the housing formed by segments 551 and 552, the socket 553 is pushed closed between the housing segments 551, 552. The socket 553 then preferably closes around the spherical end 443 of sizing device 440. When the socket 553 is fully retracted, the stem 442 of sizing device 440 is rigidly fixed perpendicular or normal to the longitudinal axis of elongate delivery device 510. When the socket 553 is partially extended, the spherical end 443 of sizing device 440 is still retained by the socket 53, but the stem 442 is able to flex freely to be parallel or perpendicular to the longitudinal axis of the elongate delivery device 510, or to any shape provided by shape memory or malleability of stem 442. When the socket 553 is fully extended, the spherical end 443 of sizing device 440 may be released from the socket 553, and the elongate delivery device 510 may be removed from the surgical field, allowing for better visualization of the device 440 placement prior to selecting a size. Other alternative releasable engagement configurations to the socket 553 are also contemplated. For example, the sizing device 440 could instead be engaged and released by an electromagnet. The socket 553 could instead be replaced by a ferrous head, for example, that changes polarity with actuation allowing for the release or engagement of the sizing device 440.

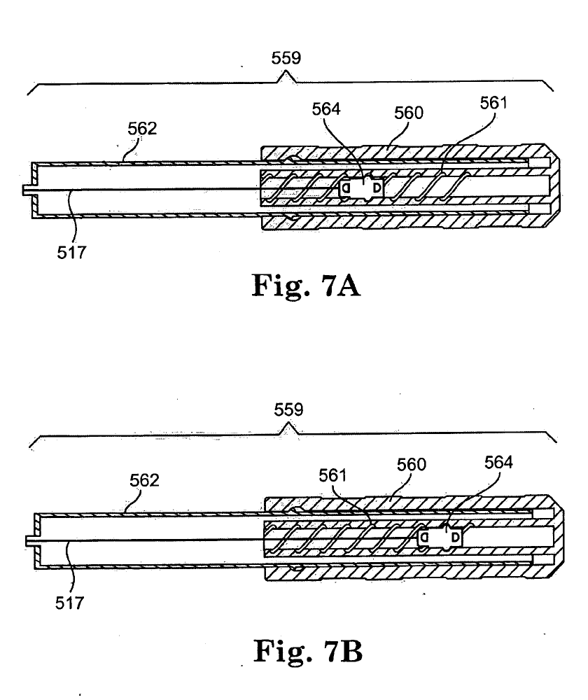

FIGS. 7A and 7B show a cross-sectional view of the proximal end 511 of the delivery device 510 in two configurations. The figures show one exemplary means for controlling the grasping means 550 of the delivery device 510 (not shown in FIGS. 7A and 7B), so as to allow the grasping means 550 to engage and retract a sizer or holder device. As shown, the handle 559 includes a rotating handle actuator 560 that may be rotated with respect to a handle head 562, which causes an inner element 564 to move distally and proximally within threads 561 in the rotating handle actuator 560. FIG. 7A shows the inner element 564 in a more distal position before rotation of the rotating handle actuator 560, and FIG. 7B shows the inner element 564 in a more proximal position. The wire 517 (preferably a single axial wire) running through the elongate segment 516 of the delivery device 510, is attached to inner element 564. The wire 517 pulls on the socket 553 when the inner element 564 is moved proximally in such a way to cause the grasping means 550 to engage the sizer device 440 and retract the sizer device 440. The wire 517 may also be pushed distally by movement of inner element 564, which causes the socket 553 to move distally and to release the sizer device 440. This is one exemplary way of remotely controlling the grasping means 550, but other configurations are also contemplated.

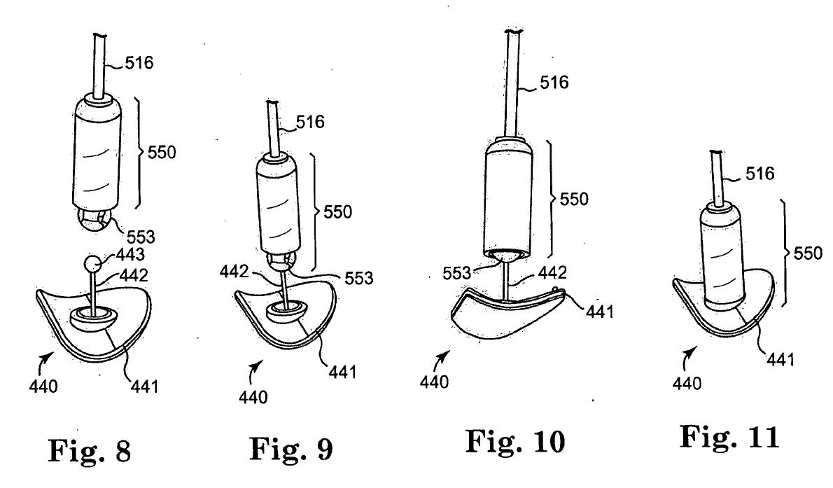

The grasping means 550 preferably has three configurations that allow a sizer device or other device, to be engaged, partially retracted or fully retracted. FIGS. 8-11 show four progressive illustrations of attachment of grasping means 550 and retraction of sizer device 440.

FIG. 8 shows the grasping means 550 in close proximity to the sizing device 440. The socket 553 is extended distally. Next in the progression, FIG. 9 shows socket 553 surrounding the spherical end 443 of the sizer device 440. The grasping means 550 is preferably controlled from the proximal end of the delivery device, as described above with regard to FIGS. 7A and 7B. The next illustration in the progression, FIG. 10, shows the socket 553 being retracted proximally into housing formed by housing segments 551, 552. In the configuration shown in FIG. 10, if the stem 442 of the sizer device 440 is malleable or jointed, as discussed above, it is possible that it may be flexed or bent while in the configuration shown. The fourth illustration, FIG. 11, shows the socket 553 retracted inside the housing formed by housing segments 551, 552, so as to not be visible. The grasping means 550 is also shown near or in close contact to the sizing plate 441 of the sizer device 440.

As described above, the delivery device 510 preferably engages the sizer device 440 (or other sizer or holder devices having a spherical portion on a stem portion) by rotation of the rotating handle actuator 560 with respect to the handle head 562, as shown in FIGS. 7A and 7B.

The various embodiments of sizer devices, holder devices and delivery devices described herein may be used together to make additional embodiments of the present invention. Additionally, certain features of the devices may be combined differently to form further embodiments of the present invention.

It is to be understood that while particular embodiments of the invention have been illustrated for use in typical valve repair procedures, various modifications to shape, and arrangement of parts can be made as may be desirable for varying applications as may relate to valve sizes or later developed techniques. The invention should not be considered limited to the specific methods and devices precisely described herein. On the contrary, various modifications will be apparent to those of ordinary skill upon reading the disclosure. Although certain embodiments are described with reference to the mitral valve, use with other valves or anatomical structures is also contemplated. The foregoing detailed description has been given for clarity of understanding only. No unnecessary limitations are to be understood there from. The entire disclosure of any article, patent or patent application identified herein is hereby incorporated by reference.

1. A device for delivering a valve annulus sizer or annuloplasty device holder to a heart valve annulus, the device comprising:
an elongate shaft comprising a proximal end, a distal end and at least a portion of the shaft being malleable,
wherein the distal end of the elongate shaft connects to the sizer or holder.

2. The device of claim 1, wherein the shaft being malleable allows the shaft to obtain a bent configuration that allows the sizer or holder to be delivered to the valve annulus through a minimal access port in a patient's body.

3. The device of claim 1, wherein the shaft has a varying cross-section with the malleable portion having a decreased cross-section.

4. The device of claim 1, wherein the malleable portion of the shaft comprises a malleable material.

5. The device of claim 4, wherein the malleable material comprises Nitinol™.

6. The device of claim 1, wherein the malleable portion of the shaft is at or near the distal end.

7. A sizer device for sizing a heart valve annulus, the device comprising:
a sizing plate; and
a stem connected to the sizing plate provided for connection of the sizer device to a delivery device, wherein the stem is bendable.

8. The sizer device of claim 7, wherein the stem comprises a malleable material.

9. The sizer device of claim 7, wherein the stem comprises a joint.

10. A holder device for holding an annuloplasty device for delivery to a heart valve annulus, the device comprising:
a holding plate; and
a stem connected to the holding plate provided for connection of the holder device to a delivery device, wherein the stem is bendable.

11. The holder device of claim 10, wherein the stem comprises a malleable material.

12. The holder device of claim 10, wherein the stem comprises a joint.

13. A delivery device that releasably attaches a sizer device or a holder device for delivery to and/or from a heart valve annulus, the device comprising:
an elongate shaft having a proximal end and a distal end; and
a socket extendible from and retractable into the distal end of the shaft, wherein the socket is extended from the distal end of the elongate shaft in order to release or surround the sizer device or the holder device and the socket is retracted into the distal end of the elongate shaft in order to attach the sizer device or the holder device to the delivery device.

14. The delivery device of claim 13, wherein the socket may be partially retracted into the distal end of the elongate shaft in order to attach the sizer device or the holder device.

15. The delivery device of claim 14, wherein the sizer device and the holder device comprise a flexible or malleable stem connected to a sizing or holding plate, and while partially retracted into the distal end of the elongate shaft, the stem of the sizer device or the holder device is able to flex or bend.

16. A delivery device that releasably attaches a sizer device or a holder device for delivery to and/or from a heart valve annulus, the device comprising:
an elongate shaft having a proximal end and a distal end, and a wire running there through;
a handle attached to the proximal end of the shaft that is attached to the wire, wherein rotation of the handle moves the wire proximally and distally; and
a socket moveable within the shaft and extendible from the distal end of the shaft and connected to the wire, wherein when the wire moves distally the socket moves distally and extends from the delivery device and when the wire moves proximally the socket also moves proximally and grasps a holder device or sizer device in order to attach the holder or sizer device to the delivery device.

17. The delivery device of claim 16, further comprising a housing that houses the socket when the wire is moved proximally.

18. The delivery device of claim 17, wherein when the socket is moved proximally, the housing surrounds the socket and causes the socket to grasp the holder device or sizer device.

19. The delivery device of claim 16, further comprising a spring connected to the socket and surrounding the wire that bias the socket to a proximal position.

20. The delivery device of claim 16, wherein the handle comprises an interior including threads, and an inner element that is attached to the wire such that rotation of the handle causes the inner element to move distally or proximally within the threads which causes the wire to move distally and proximally, respectively.
